(19) United States
(12) Patent Application Publication
(10) Pub. No.: US 2023/0357684 A1
(43) Pub. Date: Nov. 9, 2023

(54) 3D-PRINTED MODULAR MICROCHIP WITH AN INTEGRATED IMPELLER PUMP TO MODEL INTER-ORGAN COMMUNICATION

(71) Applicant: University of Virginia Patent Foundation, Charlottesville, VA (US)

(72) Inventors: Rebecca Rose Pompano, Charlottesville, VA (US); Sophie Reed Cook, Charlottesville, VA (US)

(21) Appl. No.: 18/027,202

(22) PCT Filed: Sep. 20, 2021
(86) PCT No.: PCT/US2021/051064

§ 371 Date: Mar. 20, 2023

Related U.S. Application Data
(60) Provisional application No. 63/080,320, filed on Sep. 18, 2020.

(51) Int. Cl. C12M 3/00; C12M 3/06; C12M 1/42

(52) U.S. Cl. CPC C12M 21/08; C12M 23/16; C12M 23/44; C12M 23/46; C12M 35/06

(57) ABSTRACT

The presently disclosed subject matter provides devices, systems, and methods for model inter-organ communication. In some embodiments, a multi-organ-on-a-chip (MOC) system can include one or more micro-culture well configured to receive a live tissue sample therein and an impeller-based pump in fluid communication with the one or more micro-culture well. In this arrangement, the impeller-based pump can be configured to generate fluid flow through the one or more micro-culture well.

### RELATED APPLICATIONS

The presently disclosed subject matter claims the benefit of U.S. Provisional Patent Application Ser. No. 63/080,320 filed Sep. 18, 2020, the disclosure of which is incorporated herein by reference in its entirety.

### STATEMENT OF GOVERNMENT INTEREST

This invention was made with government support under Grant No. AI131723 awarded by The National Institutes of Health. The government has certain rights in the invention.

### TECHNICAL FIELD

The subject matter disclosed herein relates generally to systems and methods for studying inter-organ communication by co-culturing 3D cell cultures, organoids, or tissue slices in recirculating media. More particularly, the subject matter disclosed herein relates to multi-organ-on-a-chip (MOC) systems and methods for operating such system.

### BACKGROUND

Within the human body, organs are in constant communication via blood and lymphatic circulation to help maintain homeostasis and to respond to external cues.² Inter-organ cross-talk occurs through the release of bioactive molecules, like cytokines, that enable intercellular communication.² These small signaling molecules get diluted in the blood and lymph fluid, making it hard to discern where they came from and where they are going. This can be especially challenging when studying specific inter-organ communication in vivo. Through the use of microfluidics, multi-organ-on-a-chip (MOC) systems can separate out these complex interactions and study them directly by connecting specialized tissue culture microenvironments via perfusion channels.³ These MOC devices can be used to culture cells,⁴⁻⁶ organoids,^{7,8} or tissue slices^{9,10} in settings that mimic physiologically relevant conditions in a quantifiable way.¹¹

Conventionally, the devices used to study inter-organ communication tend to have fabrication processes that are complex and time-consuming, while limiting the architecture of the device. Soft lithography is a traditional microfluidic fabrication method that is widely used because it is inexpensive and easy to use, but these polydimethylsiloxane (PDMS) devices are limited to layered devices and the fabrication process is lengthy and hard to scale up for mass production.^{9,12} As devices have increased in complexity, as is common with MOC systems, the reproducibility with soft lithography greatly decreases.^{9,13} Other PDMS devices include additional materials like PMMA or glass, further increasing the complexity, cost, and fabrication time.^{13, 14} Micro-machining is another fabrication technique that is highly reproducible, but the resulting devices and their respective real-world connections are incredibly complex and quite expensive to produce.³

One major way MOCs mimic in vivo function is through control of fluid flow through the various microscale organs,¹⁹ but simple and robust platforms to control flow rate are still needed. Precise fluid control is needed to mimic the very low flow rates observed for in vivo interstitial flow under physiological (0.1-1 μm/s) and pathological (1-10 μm/s) conditions.²⁰ For example, slower physiological fluid flow rates are found in the interstitium (0.1-1 μm/s) and within lymphatic capillaries (1.4-20.4 μm/s), while faster fluid flow rates are found in the blood vessel capillaries (80-180 μm/s), the lymphatic vessels (870 μm/s, with a peak of 2200-9000 μm/s), the veins (15,000-71,000 μm/s), and the aortic artery (1,000,000 μm/s). As fluid moves, it provides nutrient and waste exchange as well as communication between organs, commonly in the form of cell recirculation. Flow also applies shear stress that impacts cellular function and viability and can result in cellular adhesion, activation, and extravasation. Thus, flow control systems for organs-on-chip must generate flow in a range of physiological and pathological flow rates, while ideally enabling transport of blood-borne cells between organs without damage. In addition to controllable flow rates, additional desirable qualities for flow control systems within OOC platforms include multiplexing capabilities, compatibility with cell culture incubators in terms of temperature output, and ability to recirculate media to enable cell circulation and communication between tissues.

Many MOC devices achieve the required low flow rates by using pumps that are large, expensive, complex, and incompatible with incubators.^{1,5,9,16,21,22} To avoid these major pitfalls of available pumping techniques, in 2008 Kimura et al. integrated a stirrer-based micropump into a PDMS-based microfluidic device.⁶ A stir bar was mounted in the center of a circular chamber, controlled by an external magnetic field generated by the spinning magnet within a hotplate.⁶ The basis of this design can be traced back to the centrifugal water pump, first patented in 1990.²³ The centrifugal water pump includes a circular chamber with a rotary impeller that has curved vanes to generate a suction force, inducing fluid flow. This pump was initially designed to recirculate and oxygenate water for fish and live bait on a fishing boat, similar to the purpose of media perfusion on a microfluidic device.²³ While the central spinning stir bar provided easy and precise flow control, the device required complex fabrication processes, was not amenable to multiplexing, and was not compatible with incubators.

Accordingly, there exists a need for devices for studying inter-organ communication that can be more readily and repeatedly manufactured, while also providing a more flexible architecture that can be adapted for a variety of desired configurations.

### SUMMARY

In one aspect, the presently disclosed subject matter provides a system to model inter-organ communication. Such a system can include one or more micro-culture well configured to receive a tissue sample therein and an impeller-based pump in fluid communication with the one or more micro-culture well. In this arrangement, the impeller-based pump can be configured to generate fluid flow through the one or more micro-culture well.

In some embodiments, the one or more micro-culture well is configured to perfuse the fluid transversely with respect to the tissue sample.

In some embodiments, the system can further include a mesh base positioned within each of the one or more micro-culture well and configured to structurally support the tissue sample thereon. The mesh base can include one or more vertical posts that extend out of the one or more micro-culture well and are configured to aid in insertion and removal of the mesh base with respect to the one or more micro-culture well. In some embodiments, the system can further include a membrane positioned between the tissue sample and the mesh base.

In some embodiments, the impeller-based pump includes an impeller positioned within a substantially circular chamber and a magnetic element coupled with the impeller. In this configuration, the impeller can be rotatable within the substantially circular chamber upon application of a magnetic field to the magnetic element to generate fluid flow within the system. A magnetic field generator can be spaced apart from the impeller-based pump, the magnetic field generator being configured to apply a rotating magnetic field to the magnetic element.

In some embodiments, each of the one or more micro-culture well is contained within a sample container having a sample container inlet and a sample container outlet and the impeller-based pump includes a pump inlet and a pump outlet. In this arrangement, each sample container and the pump module are arranged to form a single closed fluid circuit in which fluid is flowed into each respective one of the sample container inlet and the pump inlet and out of each respective one of the sample container outlet and the pump outlet. In addition, one or more fluid conduit elements can be arranged to provide fluid connections among and between the impeller-based pump and the one or more sample container.

In another aspect, the presently disclosed subject matter provides a method for modeling inter-organ communication, the method including positioning a tissue sample into each of one or more micro-culture well and pumping fluid through the one or more micro-culture well.

In some embodiments, pumping fluid through the one or more micro-culture well involves perfusing the fluid transversely with respect to the tissue sample.

In some embodiments, positioning a tissue sample into each of one or more micro-culture well involves arranging a plurality of micro-culture wells in sequence within a single closed fluid circuit.

In some embodiments, pumping fluid through the one or more micro-culture well involves positioning an impeller-based pump in fluid communication with the one or more micro-culture well, the impeller-based pump comprising an impeller and a magnetic element coupled with the impeller, and applying a rotating magnetic field to the magnetic element to rotate the impeller and generate fluid flow through the one or more micro-culture well. In some embodiments, applying a rotating magnetic field can involve activating a magnetic field generator that is spaced apart from the impeller-based pump.

In another aspect, the presently disclosed subject matter provides computer-readable storage medium having computer-executable instructions stored thereon which, when executed by one or more processors, cause one or more computers to perform a method for modeling inter-organ communication, the method comprising regulating the operation of a impeller-based pump to pump fluid through a tissue sample positioned in each of one or more micro-culture well.

Accordingly, it is an object of the presently disclosed subject matter to provide a method and system to model inter-organ communication. An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIG. 1 is a plan view of a microfluidic device according to an embodiment of the presently disclosed subject matter;

FIG. 2A is a side perspective view of an impeller-based pump of a microfluidic device according to an embodiment of the presently disclosed subject matter;

FIG. 2B is a side perspective view of a sample container of a microfluidic device according to an embodiment of the presently disclosed subject matter;

FIG. 2C is a side perspective view of a straight channel fluid conduit module of a microfluidic device according to an embodiment of the presently disclosed subject matter;

FIG. 2D is a side perspective view of a fluid conduit return module of a microfluidic device according to an embodiment of the presently disclosed subject matter;

FIGS. 3A through 3D are plan views of different modular configurations of a microfluidic device according to an embodiment of the presently disclosed subject matter;

FIG. 4 is a side perspective view of a coupling mechanism for modules of a microfluidic device according to an embodiment of the presently disclosed subject matter;

FIGS. 5A and 5B are a perspective side view and side cutaway view, respectively, of a sample container of a microfluidic device according to an embodiment of the presently disclosed subject matter;

FIGS. 6A and 6B are a side perspective view and a top view, respectively, of a mesh base for use with a sample container of a microfluidic device according to an embodiment of the presently disclosed subject matter;

FIGS. 7A and 7B are a side perspective view and a top view, respectively, of an impeller-based pump of a microfluidic device according to an embodiment of the presently disclosed subject matter;

FIG. 8A is a graph showing a speed of an impeller-based pump of a microfluidic device in response to a given voltage input according to an embodiment of the presently disclosed subject matter;

FIGS. 8B and 8C are graphs showing a speed of an impeller-based pump of a microfluidic device over extended operating periods according to embodiments of the presently disclosed subject matter;

FIGS. 9A and 9B are views of different impeller configurations for an impeller-based pump of a microfluidic device according to an embodiment of the presently disclosed subject matter;

FIG. 10A is a side perspective view of an impeller-based pump of a microfluidic device according to an embodiment of the presently disclosed subject matter;

FIGS. 10B through 10E are graphs showing fluid velocities obtained from different designs of the impeller-based pump of a microfluidic device according to an embodiment of the presently disclosed subject matter;

FIG. 11A is a graph showing fluid velocity for two fluid flow regimes associated with different channel sizes according to an embodiment of the presently disclosed subject matter;

FIG. 11B is a graph showing a comparison between measured fluid velocities according to an embodiment of the presently disclosed subject matter and computational model results;

FIG. 11C is a graph showing resin cytotoxicity after a 4 hour culture according to an embodiment of the presently disclosed subject matter;

FIGS. 11D and 11E are graphs showing cell viability after 1 hour for devices with different channel sizes according to embodiments of the presently disclosed subject matter;

FIG. 12A is a schematic representation of a motor-based external pump platform configured for use with a microfluidic device according to an embodiment of the presently disclosed subject matter;

FIG. 12B is a graph showing a speed of an impeller-based pump of a microfluidic device in response to a given voltage input according to an embodiment of the presently disclosed subject matter;

FIG. 12C is a graph showing temperature over time of a microfluidic device using various pump configurations;

FIG. 12D is a graph showing speeds of an impeller-based pump of a microfluidic device at various operating voltage over extended operating periods according to embodiments of the presently disclosed subject matter;

FIGS. 12E through 12G are graphs showing the relationship between impeller rotation and fluid velocity for a microfluidic device according to embodiments of the presently disclosed subject matter.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a,” “an,” and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising,” which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Unless otherwise indicated, all numbers expressing quantities of size, temperature, time, weight, volume, concentration, capacitance, specific capacity, discharge capacity, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes, but is not limited to, 1, 1.5, 2, 2.75, 3, 3.90, 10 4, and 5).

### Multi-Organ-on-a-Chip (MOC) Systems

To address the issues with conventional MOC systems, in one aspect, the present disclosure provides a reconfigurable 3D-printed microfluidic device having an integrated impeller-based pump. The novel impeller-based pump integrates elements from both the historic centrifugal water pump and a stirrer-based micropump by inserting a magnetic stir bar into a 3D-printed impeller that rotates in a circular chamber within the modular chip.^{6,23} In some embodiments, the magnetic impeller is externally controlled using magnets mounted on a motor that is operable to produce controllable low speeds (i.e. flow rates) by changing the voltage the motor receives. The integrated impeller pump is inexpensive, easy to multiplex, cuts down on size relative to other conventional pumping methods, and includes few wires with little to no heat given off. In some embodiments, the device is used to gain a better understanding of the immune system by examining lymph node slice culture and how it interacts with other organs in the body. By way of example and not limitation, in some embodiments, the presently disclosed subject matter can provide communication between two or more tissue samples to create a biomimetic model of inter-organ communication.

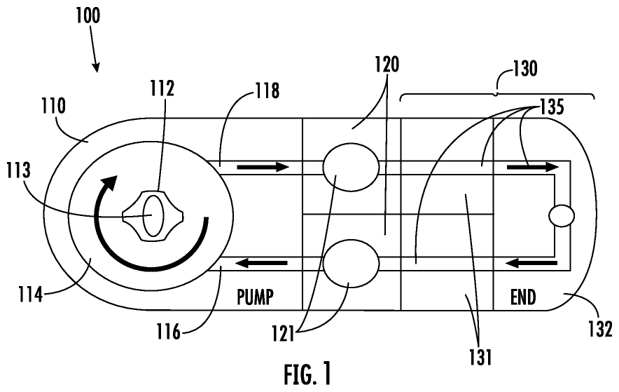

Referring to the embodiment shown in FIG. 1, a microfluidic device, generally designated 100, includes an integrated impeller-based pump 110 that can be used to simulate physiological fluid flow, and one or more sample container 120 is arranged in fluid communication with the impeller-based pump 110 and is configured to receive a tissue sample therein. In some embodiments, the tissue sample includes one or more ex vivo tissue slice or a representative model thereof. The microfluidic device 100 can further include one or more additional fluid conduit elements 130 to connect the impeller-based pump 110 and the one or more sample container 120 such that the microfluidic device 100 defines a single closed fluid circuit.

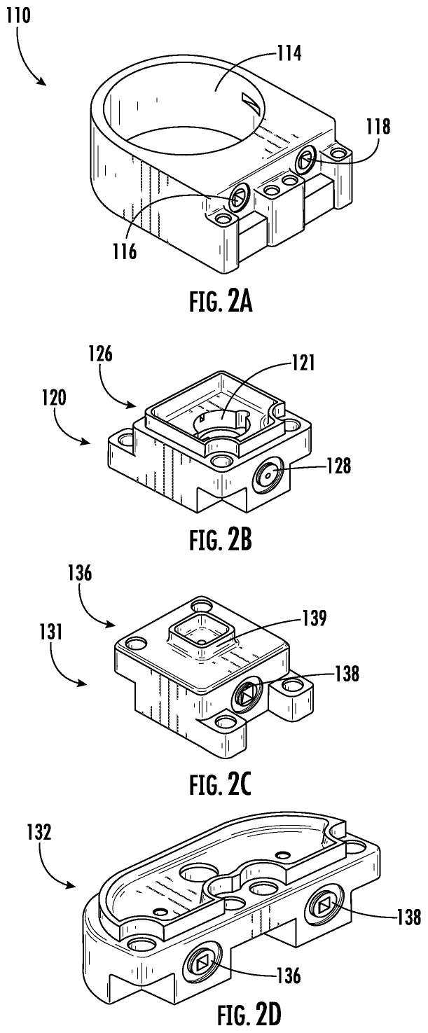

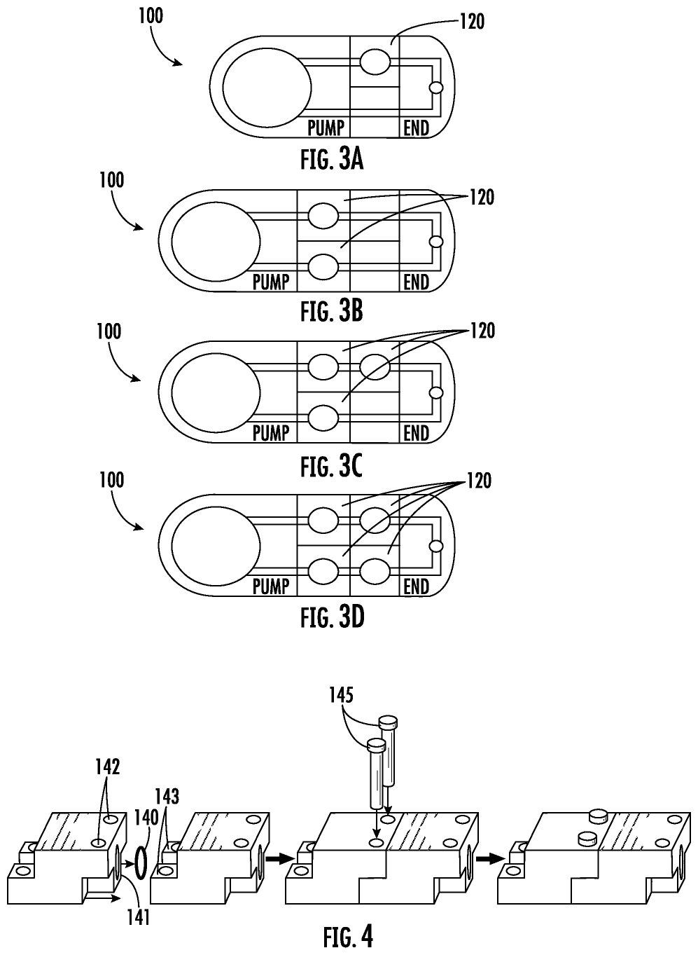

In some embodiments, the microfluidic device 100 comprises distinct modular units that can be assembled into any of a variety of configurations that can be easily reconfigured based on the needs of each specific experiment. As shown in FIGS. 2A through 2D, at least four different types of base modules can combine to provide the functionality of a MOC system. FIG. 2A shows a modular configuration for impeller-based pump 110, FIG. 2B shows a modular configuration for sample container 120, and FIGS. 2C and 2D show various configurations for fluid conduit elements 130 that are used to connect the other modules and complete the fluid circuit. Specifically, FIG. 2C shows a straight channel module 131, and FIG. 2D shows a return module 132 that redirects fluid back towards the impeller-based pump 110. In some embodiments, each straight channel module 131 and return module 132 contains a single channel 135 providing fluid communication between a channel inlet 136 and a channel outlet 138. In some embodiments, each straight channel module 131 includes a small port 139 on the top of the module to assist with filling the microfluidic device 100. Each module is designed to fit together in any of a variety of configurations, making the device completely customizable. For instance, the number of sample containers 120 connected in the device can be expanded to accommodate the culture of two, three, and four tissue slices simultaneously as shown in FIGS. 3A through 3D. In addition, in some embodiments, the straight channel modules 131 and the sample containers 120 can be configured to be interchangeable such that any of a variety of combinations of numbers and arrangements of these modules can be situated in sequence between the impeller-based pump 110 and the return module 132. Alternatively or in addition, additional modules can be incorporated to provide further functions, for example to mimic biological features, such as a membrane functioning as a blood brain barrier.

In some embodiments, the modules of the microfluidic device 100 can be produced using 3D printing techniques. For example, stereolithography (SLA) 3D printing is an additive manufacturing fabrication technique that uses photocurable resins to build up a device layer by layer.¹⁵ This process can allow for rapid prototype printing (5-60 min) and to design and print intricate architecture within a single 3D structure.^{12,16-18} When applied to the microfluidic device 100 discussed herein, the use of stereolithography 3D printing can greatly reduce the fabrication time, increase reproducibility, and make the device easy to use and share with collaborators. That being said, while 3D printing is an easy way to reproducibly fabricate microfluidic devices with complex architecture in a short period of time, the liquid photopolymer resins used for stereolithography (SLA) and digital light processing (DLP) 3D printing are often cytotoxic. In addition, the use of additives such as optical absorbers and plasticizers can enhance the print resolution, enabling smaller internal channel sizes and smaller port diameters, such as with the MiiCraft BV007a resin, but these may result in increased toxicity if these molecules leach out of the device. Pre-leaching the printed parts is one way to reduce cytotoxicity, as is coating with impermeable protective materials such as parylene C. Alternatively, as the impeller pump platform is intended to recirculate fluid and cells within MOC systems and other biological model systems, the material used to fabricate the device and impeller can be selected to be cytocompatible for the timescale of the experiment, similar to polydimethylsiloxane (PDMS) that is used in traditional soft lithography microfabrication techniques. Fabrication in well-characterized cell-culture materials such as polystyrene, polypropylene, acrylic, or cyclic olefin copolymer is feasible by methods such as machining, hot embossing, or extrusion, paired if needed with acrylate or silicone adhesives for multilayer parts.

In one exemplary situation, a prototype modular device was designed using Fusion 360 drawing software and 3D printed using a MiiCraft Ultra 50 SLA Printer with BV007a clear resin (MiiCraft) and FormLabs Clear. Although existing modular microfluidic devices^{16,21,22} can be composed of independent units that have a particular pattern of channels with a specific function, the device is distinguished by its inclusion of a tissue module to accommodate live tissues or models thereof, among other features. For example, the printed modules were designed to have sufficient tolerance at the connections of each piece to fit together tightly. For example, the internal channel dimensions were optimized to be 500×500 μm, the smallest dimensions that could be printed reproducibly within each module with this combination of printer and resin.

Whether using additive manufacturing or some other fabrication technique, the connections between the modules can be designed to be sealed tightly to provide fluid circulation throughout the microfluidic device 100 without any leaks. As shown in FIG. 4, such a water-tight seal can be generated by placing an O-ring 140 in a shallow well 141 that surrounds the channel outlet of each of the module units. The shallow well 141 on each module can further be coated with a hydrophobic substance (e.g., a silicone polymer commercially available under the trademark Rain-X, or a fluorinated silane) to increase the hydrophobicity of the printed pieces. Each module can further include a set of upper mounting openings 142 on one side of the module (e.g., at the channel outlet) and a set of lower mounting openings 143 on an opposing side of the module (e.g., at the channel inlet). Once the modules are fitted together, the upper mounting openings 142 of a first module can be aligned with the lower mounting openings 143 of a second module, and pins 145 can be inserted therethrough to fix the modules together. This coupling arrangement can generate sufficient pressure on the O-ring to effectively seal the connection. This connection method is similar to a mortise and tenon joint, which is a type of joint that is generally used to connect two pieces without glue or screws and is commonly seen in wood-working.²⁴ In some embodiments, this form of joint comprises two parts: the mortise (upper and lower openings 142 and 143) and the tenon (pin 145). Those having ordinary skill in the art will recognize, however, that any of a variety of other mechanisms can be used by which the modules can be coupled together with substantially fluid-tight conduit connections.

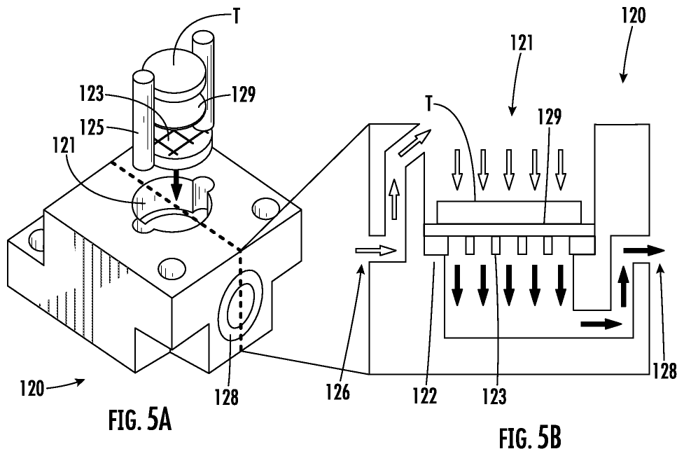

Regarding the particular configuration of the individual component elements, in some embodiments, the sample container 120 can be optimized to house ex vivo tissue slices in a user-friendly way. In particular, for example, referring to the configuration shown in FIGS. 5A and 5B, the sample container 120 can be designed to accommodate long-term culture of thick slices of live tissue T, rather than more traditional cell cultures. In this regard, in some embodiments, the sample container 120 includes a micro-culture well 121 that is configured to perfuse flow over the slice of tissue T in applications in which such an interaction is preferred. Alternatively, in some embodiment, the micro-culture well 121 is configured to perfuse flow substantially perpendicularly to the slice of tissue T. For example, in the embodiment shown in FIGS. 5A and 5B, a sample container inlet 126 can be arranged in communication with a top of the micro-culture well 121, a sample container outlet 128 can be arranged in communication with a bottom of the micro-culture well 121, and the slice of tissue T can be arranged between the sample container inlet 126 and the sample container outlet 128. This so-called transverse perfusion provides biomimetic interstitial flow and shear stress to the tissue T and helps slowly-diffusing signaling molecules access the center of the sample (e.g., allowing sampling of a tissue slice of 100-1000 μm thick). In some embodiments, the width/diameter of the micro-culture well 121 is much larger (e.g., 7 mm) compared to diameters of the sample container inlet and outlet 126 and 128 (e.g., 0.5 mm-1 mm) such that the fluid flow velocity is 150-fold lower in the relatively larger space. Even with this decrease, however, the impeller pump design can be selected as discussed below to provide initial fluid velocities on the scale of mm/s.

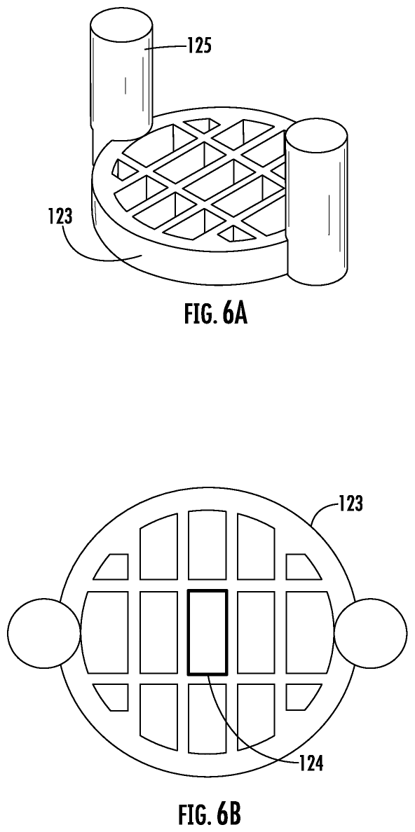

Referring in particular to FIGS. 5A, 5B, 6A, and 6B, to reduce the potential for damage to the tissue T when inserting and removing tissue slices, particularly when sampling thick slices as discussed hereinabove, a mesh base 123 can be removably positioned within and/or integrated into the micro-culture well 121 of the sample container 120. The mesh base 123 can provide structural support for the slice of tissue T, but the mesh base 123 can include a plurality of openings 124 as shown in FIGS. 6A and 6B to allow free flow of fluid that perfuses through the tissue T. In some embodiments, the mesh base 123 rests on a ledge 122 within the micro-culture well 121 in the sample container 120, and the mesh base 123 can include vertical posts 125 that extend up above the walls of the micro-culture well 121 to allow for easy insertion and removal. This design limits the handling of the delicate slices, while also reducing the potential for leaks within the device when adding or removing a tissue sample. In addition, in some embodiments, a membrane 129 can be placed between the slice of tissue T and the mesh base 123 to provide further mechanical support for the tissue T. By choosing the size of the openings 124 in the mesh base 123 and/or the pore size of the membrane 129, it is possible to selectively prohibit or enable the circulation of cells between culture chambers on the microfluidic device 100. The membrane may comprise any porous membrane, including track-etched, woven, or spun membranes made from polycarbonate, polyester, vinyl, nylon, or metal. In some embodiments, the membrane can be patterned to allow flow through only one portion or region of the container. For example, a porous membrane may be backed by with an additional non-porous support beneath, such as a transparency sheet, with a hole cut in one or more regions. Alternatively, the membrane may be patterned by micropatterning, photolithography, or stamping, to permit fluid flow only through certain regions.

As discussed above, fluid flow is provided to the one or more sample container 120 by the impeller-based pump 110. Referring again to FIGS. 1 and 2A, in some embodiments, the impeller-based pump 110 includes an impeller 112 inserted into a substantially circular chamber 114 that has both a pump inlet 116 and a pump outlet 118. A magnetic element 113 can be inserted into or otherwise coupled with the impeller 112, and a magnetic field applied to the impeller-based pump 110 can thereby cause the impeller 112 to rotate. In this arrangement, as the impeller 112 is rotated, the impeller-based pump 110 generates fluid flow within the microfluidic device 100. Based on the orientation of the magnetic element 113 within the impeller 112, the impeller-based pump 110 can be configured to drive flow in either a clockwise or counterclockwise direction.

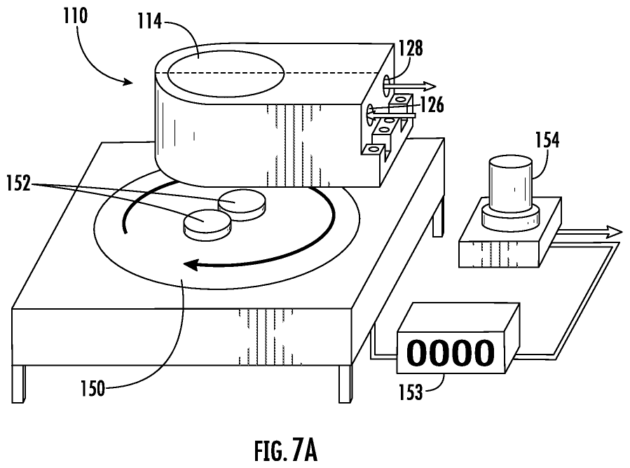

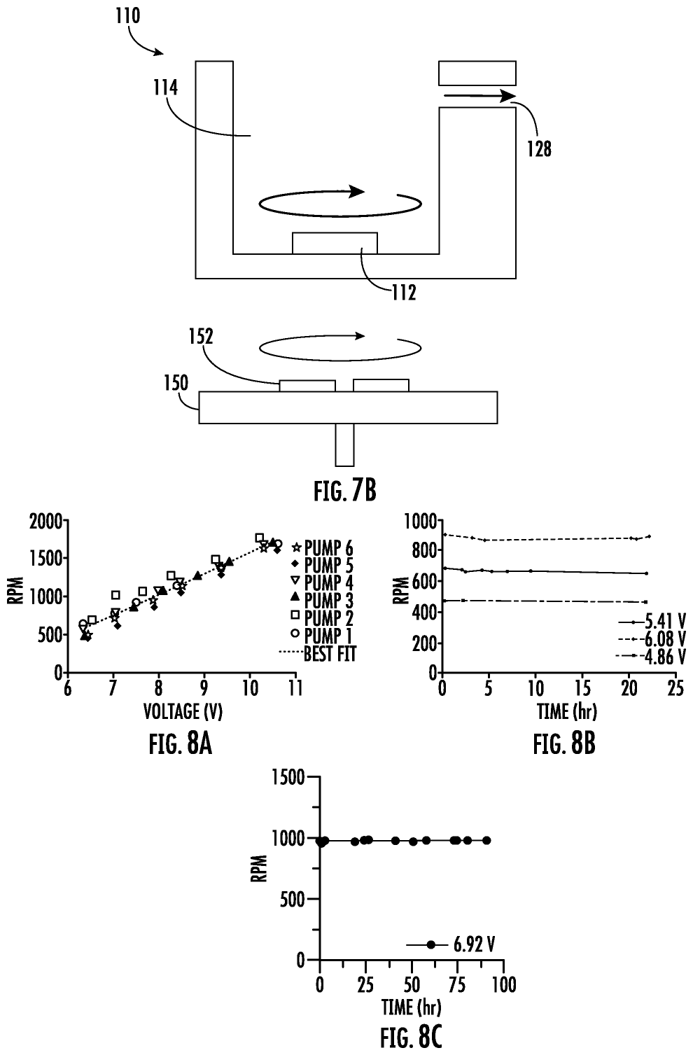

Referring to FIGS. 7A and 7B, for example, the impeller-based pump 110 can be arranged in proximity to magnetic field generator 150 that is configured to generate a rotating magnetic field for causing rotation of the impeller 112. In some embodiments, for example, the magnetic field generator 150 can be a motor that has one or more magnets 152 mounted for rotation together. In any configuration, the operation of the magnetic field generator 150 is able to drive the impeller-based pump 110 inside the microfluidic device 100 without complex wiring or pneumatic controls. In addition, this externally-driven pump design is inexpensive, easy to build, does not generate a significant amount of heat like many traditional pumps, and has multiplexing capabilities. In some embodiments, the motor 150 is a variable-speed device that is controllable (e.g., by changing the voltage applied to the motor 150) to regulate the speed of rotation of the impeller 112 and thus regulate the flow rate of the fluid discharged by the impeller-based pump 110. In the configuration shown in FIG. 7A, for example, the motor 150 is connected to a voltmeter 153 and potentiometer 154 to provide a voltage readout and control, respectively.

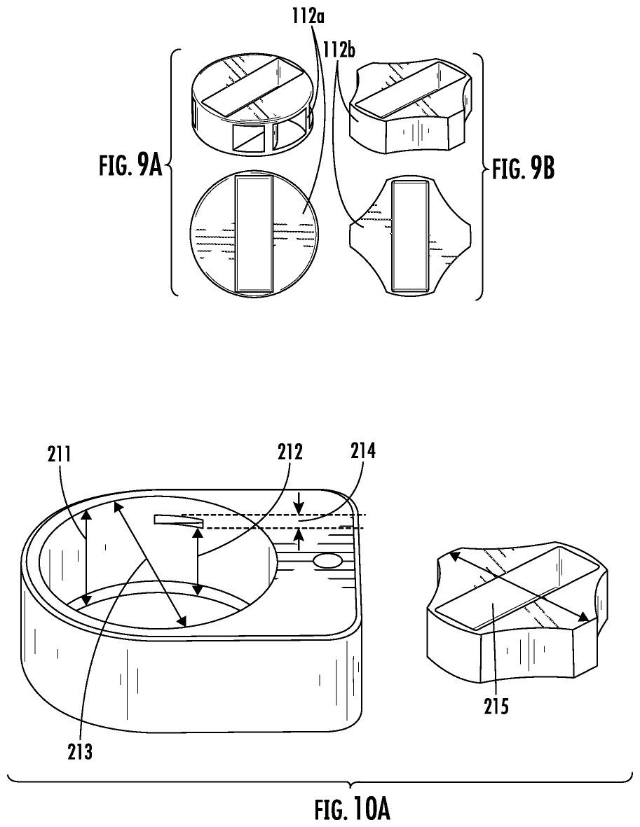

As shown in FIG. 8A, the RPMs of the impeller 112 in an example configuration are shown at various voltages. In this example, the RPMs for each of six externally-driven impeller-based pumps 110 were quantified at increasing voltages, showing a positive linear relationship as expected. To provide the desired functionality for MOC systems, the microfluidic device 100 can be designed to culture tissue slices overnight, so the flow rate needs to be able to stay relatively constant during that length of time. The RPM stability for the first pump platform for an example embodiment is shown in FIG. 8B over a period of 22 hours at a low, medium, and high voltage. As shown in FIG. 8B, there was little to no change in the RPMs over this period of time, meaning the impeller-based pump 110 can be stable for overnight culture use.

In some embodiments, precise flow rate control is valuable in an MOC device to properly mimic both physiological (0.1-1 μm/s) and pathological (1-10 μm/s) environmental conditions.²⁰ To achieve more biomimetic flow velocities, the design of the impeller-based pump 110 and the impeller 112 positioned therein can be configured to control the fluid flow rate within the device. In some embodiments, the impeller 112 can have a “closed impeller” configuration 112a shown in FIG. 9A, which closely resembles the efficient impellers found in centrifugal water pumps.²³ Such a closed impeller configuration 112a can have curved vanes, which allows the impeller 112 to generate higher suction power within the substantially circular chamber 114 (see also FIGS. 7A and 7B) that leads to an increase in the fluid velocity. Alternatively, in other embodiments, the impeller 112 can have a substantially cross-shaped configuration 112b as shown in FIG. 9B that is designed to be comparatively inefficient, generating suction mostly from friction within the impeller-based pump 110. The pump well and impeller dimensions were optimized to reduce the velocity throughout the device. The pump well was greatly increased in depth and diameter relative to the impeller. Under these fully optimized pump module conditions, the closed impeller configuration 112a can result in a velocity of 6.4 μm/s through the tissue and the cross-shaped impeller configuration 112b can result in a velocity of 0.5 μm/s through the tissue. These different impeller configurations can thus be used for different applications, for both physiological and pathological fluid flow conditions.

To fully achieve a fluid velocity that was within the correct physiological range, the pump well dimensions can be configured alongside the impeller design. When designing the dimensions of the pump well, impeller, and entry points of the microchannel, it can be assumed that the hydrodynamic energy produced in the pump well by the rotating impeller decreases further from the impeller, such as due to viscous energy losses. Consistent with this principle, it was observed that if the impeller filled the majority of the cross-sectional area of the well, then the hydrodynamic energy generated fluid velocities that were able to reach as high as 33,000 μm/s, sufficient to model venous or arterial fluid flow. In contrast, where it is desired to model slower capillary and lymphatic vessel flow velocities, a large well diameter (e.g., about 26 mm) can be chosen relative to the width of the impeller piece (e.g., about 11.5 mm). Similarly, the intersection height of the channels approximately two-thirds up the side of the well (e.g., about 8.5 mm from the base of a 12-mm deep well) can be designed to slow the flow rates through the corresponding channel compared to a lower intersection height. In this manner, a 3-dimensional architecture for the device and impeller can be achieved and optimized through rapid 3D printing, enabling the pump to attain biologically-relevant fluid flow regimes.

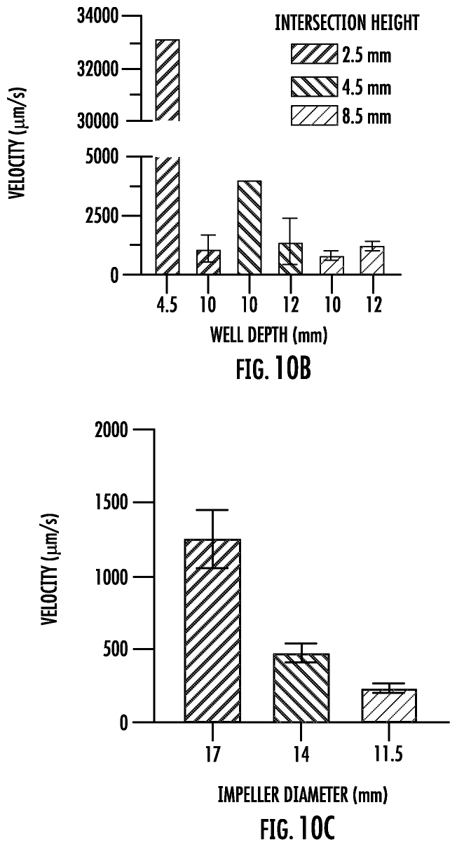

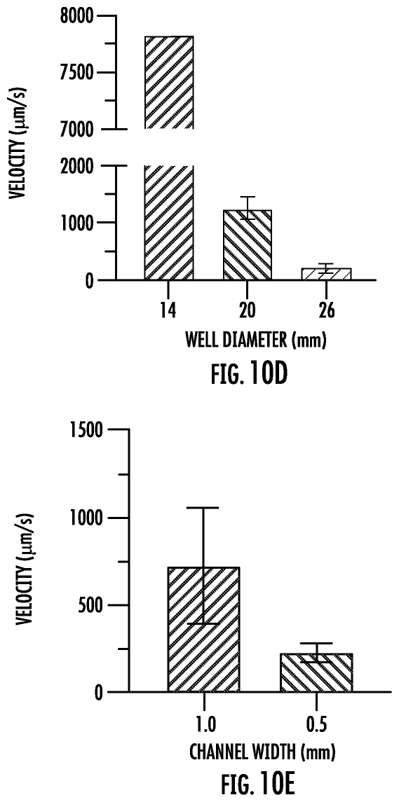

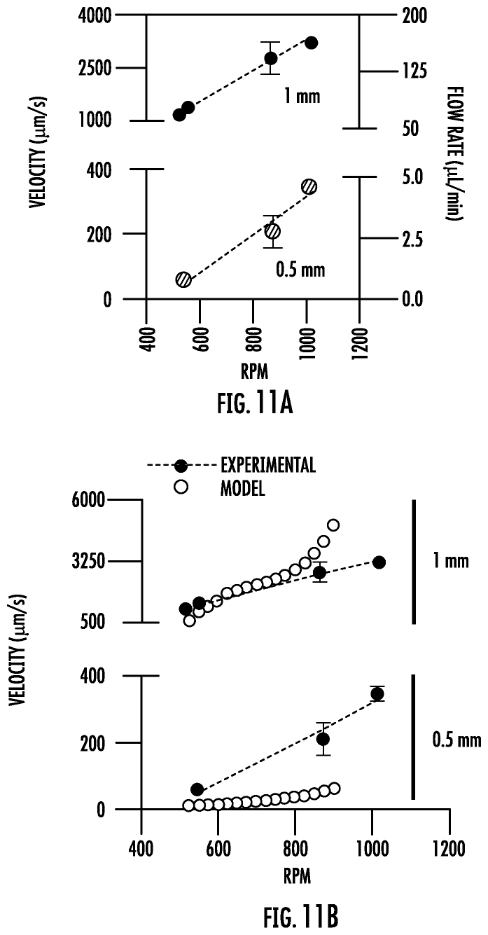

As shown in FIGS. 10B through 10F, for example, modifying the different parameters of the impeller-based pump 110 shown in FIG. 10A can result in different fluid velocities. As shown in FIG. 10B, velocity can be controlled as a function of well depth 211 and intersection height 212 with the well diameter 213 (e.g., about 20 mm), channel width 214 (e.g., about 1 mm), and impeller diameter 215 (e.g., about 17 mm) held constant. As shown in FIG. 10C, velocity can be controlled as a function of impeller diameter 215 with the channel wall intersection height 212 (e.g., about 8.5 mm), well depth 211 (e.g., about 12 mm), well diameter 213 (e.g., about 20 mm), and channel width 214 (e.g., about 1 mm) held constant. As shown in FIG. 10D, velocity can be controlled as a function of well diameter 213 with the channel wall intersection height 212 (e.g., about 8.5 mm), well depth 211 (e.g., about 12 mm), channel width 214 (e.g., about 1 mm), and impeller diameter 215 (e.g., about 11.5 mm) held constant. Finally, as shown in FIG. 10E, velocity can be controlled as a function of channel dimensions 214 with the channel wall intersection height 212 (e.g., about 8.5 mm), well depth 211 (e.g., about 12 mm), wall diameter 213 (e.g., about 26 mm), and impeller diameter 215 (e.g., about 11.5 mm) held constant. It is notable, for example, that a channel size of about 0.5 mm results in a low velocity range (e.g., between about 60-350 μm/s) that is comparable to blood vessel capillaries in vivo, whereas a channel size of about 1 mm results in a higher velocity range (e.g., between about 1160-3200 μm/s) that is comparable to lymphatic vessels in vivo.

Despite the increasing pool of innovative organ-on-a-chip platforms, many are designed for cell or organoid culture rather than tissue slices and tend to be specifically tailored for each lab's specific application. In contrast, the modular device and impeller-based pump platform proposed herein is not only user-friendly and customizable, but it is completely accessible to other researchers focusing on inter-organ communication, especially between tissue slices and also, optionally, between 3D cultures in hydrogels or other supportive 3D matrices. Incorporation of tissue slices and 3D cultures is made particularly user-friendly by incorporation of the transwell-style inserts that can be added and removed from the device as needed, e.g. for imaging on a microscope or for switching the tissue sample mid-experiment. An aspect of the presently disclosed subject matter provides, among other things, the innovative of on-board impeller pump that provides continuous recirculating flow, without complex pneumatics, dynamic electronic triggering, or undesirable heat output. Having the pump on-board the MOC shortens the pathlength for fluid flow compared to running tubing to-and-from an external pump system, particularly as many external pumps must be located outside of the cell-culture incubator. This MOC platform can be used to study, among other things, physiological and pathological lymph node function with models of the central nervous system (blood-brain barrier and brain), gut, tumor, and more. In the future, this device can be used to focus on inflammation in the central nervous system and how it relates to autoimmune diseases.

An aspect of an embodiment of the present disclosure includes, but not limited thereto, a computer readable medium having computer-executable instructions stored thereon which, when executed by one or more processors, cause one or more computers to perform a method for modeling inter-organ communication. In this regard, in some embodiments, the impeller-based pump 110 can be computer-controlled to efficiently regulate the rotation of the impeller 112 and thus the fluid flow rate through the system.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

In one tested configuration, the device and impeller were designed using Fusion 360 and 3D printed using a CADWorks 3D MiiCraft Ultra 50 Printer with two resins, MiiCraft BV007a and FormLabs Clear. The impeller housed a magnetic stir bar and was rotated using an external platform which consisted of two magnets mounted on a computer fan. The fan rotation was controlled using a potentiometer with a voltmeter for voltage readout. A computational model of the pump was designed using computational fluid dynamics studies via ANSYS software. Fluid velocity was characterized by tracking dye moving through the device using a Dino-Lite Edge 3.0 digital microscope and the images were analyzed using

DinoXcope software. To observe cell circulation, primary murine splenocytes were stained with Calcein AM and images were captured before and after impeller rotation using a Zeiss AxioZoom macroscope. To measure cell viability, cells were cultured on-chip for 1 hr at 37° C., then stained with Calcein AM and 7-AAD for live and dead cells, respectively, and quantified using flow cytometry.

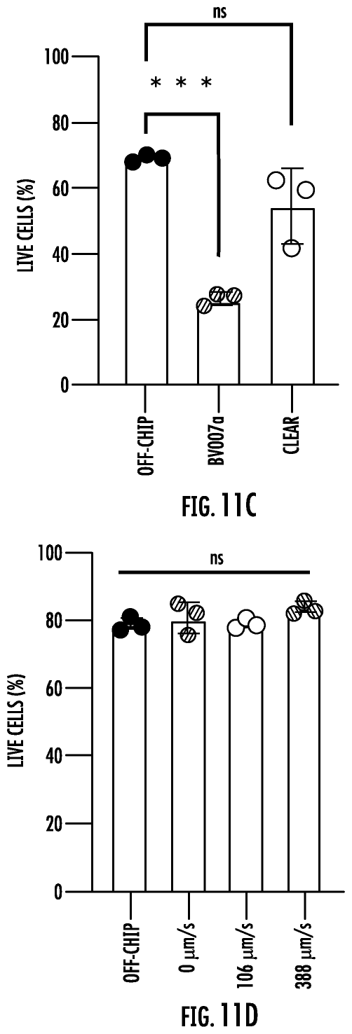

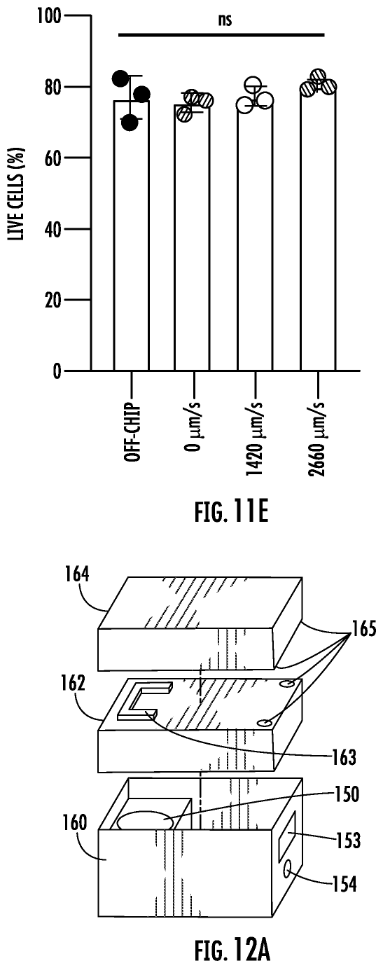

The impeller pump comprised a magnetic impeller in the center of a large well, integrated into in a 3D-printed device with a looped channel for recirculating fluid flow in an arrangement similar to that shown in FIG. 1 without any sample containers. The driving force of impeller rotation was a rotating magnetic field. This feature provided the simplicity of this platform because no tubing was required, which reduced the likelihood of leaks and further complications. Computer fans were selected to rotate a pair of magnets to drive impeller rotation in a configuration similar to that shown in FIG. 7A, as these fans do not emit heat. The external pump platform was housed in a sealed project box with two fans per box, with the small size allowing for expansion into six discreet pumps with separate voltage control for each fan. By controlling the fan speed and altering the device dimensions, the impeller pump achieved fluid flow in a low (59-346 μm/s) and high (1156-3206 μm/s) range of velocities as shown in FIG. 11A. The measured fluid velocity within the microfluidic channels was comparable to the Ansys computational model for both the 0.5 mm and 1 mm channel sizes as shown in FIG. 11B. Material cytotoxicity was determined for two different resins by culturing primary splenocytes for 4 hr in the printed devices without fluid flow. Although the BV007a resin has a higher print resolution, it caused a significant decrease in in cell viability, whereas the FormLabs Clear resin did not, as shown in FIG. 11C. To observe cells recirculating on-chip, fluorescently-labeled splenocytes were added to a pre-filled device. When there was no impeller rotation, cells settled within the well and did not enter the channel. Once the impeller began to rotate, the cells resuspended and circulated through the channels of the device. To test the impact of impeller rotation on viability, cells were circulated on-chip for 1 hr at 37° C. at low and high rotational speeds (RPMs of 387 and 814, respectively) in devices with 0.5 mm channels as shown in FIG. 11D and 1 mm channels as shown in FIG. 11E. Compared to cells cultured off-chip, there was no significant difference between any of the tested conditions, which showed that the impeller rotation does not have a significant effect on cell viability.

### Example 2

A design goal for the impeller pump was minimal heat emission to allow for extended cell culture within an incubator. Stable temperatures (±1° C.) play a role in maintaining viable cell cultures, and we previously found that a peristaltic pump rapidly raised the temperature inside a culture incubator if not countered with cooling packs. To test this feature of the impeller pump, the temperature within a cell culture incubator was monitored over time with six external pump platforms running at a high rotational speed (>10 V) for 9 hrs. During this time period, there was no change in the temperature reported by the cell culture incubator (37.0° C.), which indicated that the multiplexed external pump platform did not emit a noticeable amount of heat and thus are compatible with extended use inside the incubator.

### Example 3

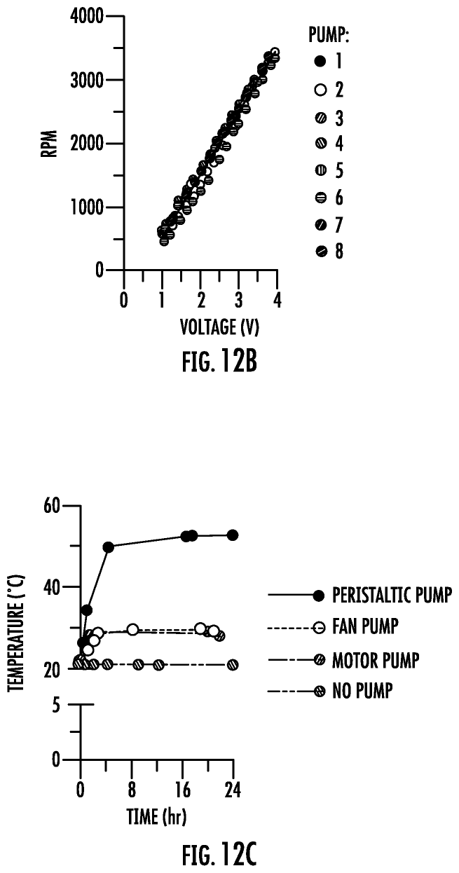

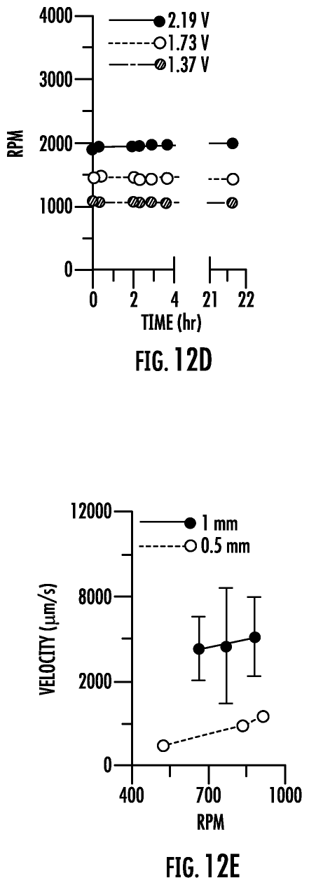

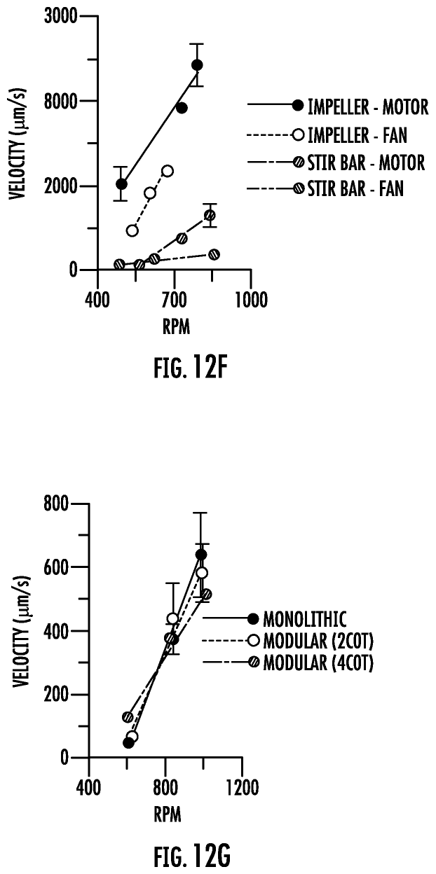

In a further example, a motor-based impeller pump platform was provided. Instead of a computer fan, a small DC motor was used to rotate the magnets, resulting in magnetic impeller rotation. The voltage the motor received was controlled by a potentiometer (POT) with a voltage readout from a voltmeter in an arrangement substantially similar to the configuration shown in FIG. 7A. As shown in FIG. 12A, the motor-based external pump platform included a base 160 that houses the motor 150, potentiometer 154, and voltmeter 153; a top 162 that encloses the electronics; and a lid 164 to cover the device while in use. The entire housing was 3D-printed using a Fused Deposition Modeling (FDM) 3D-printer. The magnets 152 are mounted on the motor 150 by gluing them into a 3D-printed magnet mount that has a built-in fan on the base to assist with cooling the motor. In addition to the motor 150, potentiometer 154, and voltmeter 153, there are also three heat sinks within the inside of the pump platform to help evenly distribute the heat within the enclosed space. When the top 162 attached to the base and sealed using hot glue, the microfluidic device 100 and impeller 112 can be placed on the top 162 in a chip holder 163. The pump platform lid 164 fits onto the top 162 using small magnets 165. The use of a motor to drive impeller rotation results in a reduced overall size, meaning the motor-based pump can run twice as many devices within the same amount of space as the fan-based pump. A modular device including one tissue module and one channel module was initially filled with red dye at t=0 min. Blue dye was added to the pump well and moved throughout the channel over a period of 17.9 min. As shown in FIG. 12B, the rotations-per-minute of the impeller scaled substantially linearly compared to the DC motor voltage for all 8 motor-based pump platforms. As shown in FIG. 12C, the change in temperature was measured within an insulated Styrofoam box as various pump methods were run over a period of 24 hrs. As shown in FIG. 12D, the impeller rotation was stable over a period of 22 hrs at three different voltages. As shown in FIG. 12E, the velocity increased as RPMs increased for both the 0.5 mm channel size and the 1 mm channel size, with the larger channel size resulting in a higher fluid flow regime, and FIG. 12F shows a comparison of the velocities measured for both the fan-based pump and the motor-based pump while using the traditional cross impeller and a 10 mm stir bar. Finally, FIG. 12G shows a comparison of the velocities across a range of RPMs for the monolithic device, the modular device with 2 channel modules (2COT), and the modular device with 4 channel modules (4COT). The velocities were all similar, showing the device modularity doesn't significantly impact the velocity.

### Example 4

A computational model was used to predict the levels of shear stress within the device during impeller-driven fluid flow. Shear stress is a major consideration for cell recirculation, as high shear stress can damage the cells and diminish viability. Physiological shear stress spans 0.6-12 dyn/cm² in lymphatic vessels and 0.35-70 dyn/cm² in normal blood vasculature. A shear stress values of 100 dyne/cm² is sometimes considered the threshold for pathological shear, which reaches >1500 dyn/cm² in diseased or stenotic vessels. Using the computational model, fluid shear stress levels during impeller rotation were estimated at various regions within the device. Looking at the impeller surface, 93.2% of the surface was <100 dynes/cm² (i.e., within the physiological range). The highest shear stress, 400 dynes/cm², was found along the edges of the impeller, although it is believed that cells suspended in the circulating media would rarely contact the impeller surface or edges due to centrifugal forces and the large volume of the pump well. Within the channels, the surface shear stress approximations were much lower and well within the physiological range: 0.04-0.10 dynes/cm² in the 0.5 mm channel, and 0.40-1.22 dynes/cm² in the 1 mm channel, with the highest stress in the corners of the channel. Based off of these results, it is predicted that the impeller rotation would not have a significant impact on the viability of circulating cells.

### Example 5

Cell recirculation is a key feature of inter-organ communication in vivo, and a new pump for organs on chip should be able to drive cell recirculation without impairing viability. The ability of the impeller pump to drive continuous white blood cell recirculation was tested under fluid velocities found within lymphatic vessels and vasculature in vivo. Given the depth and size of the pump well, it was possible that cells would settle to the bottom of the pump well instead of remaining suspended for recirculation through the microfluidic channel, especially at low RPM. To address this concern, primary splenocytes were stained with Calcein AM and deliberately allowed to settle to the base of the pump well of a pre-filled device while the impeller was off. Imaging at this time confirmed that the cells settled along the base of the pump well and that no cells were present within the channels. Once the impeller began to rotate at a low rotational speed (e.g., about 6.20 V, 419 RPM), the cells resting on the base of the pump well were resuspended and began to recirculate through the channels, where they were visible entering the reservoir. Cells moved much faster through the 1-mm channel, as evidenced by the blurring of fluorescently-labeled cells moving through the center of the reservoir, than through the 0.5 mm channel, consistent with the slower flow rate in narrower channels. Thus, the rotation of the impeller pump successfully resuspended cells even from rest and achieved continuous cell recirculation through the device.

### Example 6

The biocompatibility of the system was tested under a range of biomimetic fluid velocities. Cells were continuously recirculated through the microfluidic device (post-treated, FormLabs Clear) for 1 hr, while the whole system was inside a cell culture incubator, to provide ample time for any mechanical damage from the impeller rotation to impact cell viability. As discussed above, a 0.5 mm channel can be used to achieve low velocities similar to those measured within blood capillaries in vivo, and a 1 mm channel can be used to achieve higher velocities similar to those measured within lymphatic vessels in vivo. Compared to the off-chip control and the cells cultured on-chip with no impeller rotation, there were no significant differences in viability for the cells moving 106 μm/s, 388 μm/s, 1420 μm/s, or 2660 μm/s. It is believed that the impeller-driven micropump did not cause mechanical damage to the cells even at the higher RPM or flow velocities. Thus, the microscale impeller pump was a feasible means for cell recirculation over a wide range of biomimetic flow rates, making it suitable for future use in microscale cultures and OOCs.

In summary, while the presently disclosed subject matter has been described with respect to specific embodiments, many modifications, variations, alterations, substitutions, and equivalents will be apparent to those skilled in the art. The presently disclosed subject matter is not to be limited in scope by the specific embodiment described herein. Indeed, various modifications of the presently disclosed subject matter, in addition to those described herein, will be apparent to those of skill in the art from the foregoing description and accompanying drawings. Accordingly, the presently disclosed subject matter is to be considered as limited only by the spirit and scope of the disclosure (and claims) including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

### REFERENCES

The following patents, applications and publications as listed below and throughout this document are hereby incorporated by reference in their entirety herein.
1. Satoh, T. et al. A multi-throughput multi-organ-on-a-chip system on a plate formatted pneumatic pressure-driven medium circulation platform. Lab. Chip 18, 115-125 (2018).
2. Castillo-Armengol, J., Fajas, L. & Lopez-Mejia, I. C. Inter-organ communication: a gatekeeper for metabolic health. EMBO Rep. 20, (2019).
3. Edington, C. D. et al. Interconnected Microphysiological Systems for Quantitative Biology and Pharmacology Studies. Sci. Rep. 8, 4530 (2018).
4. Xiao, S. et al. A microfluidic culture model of the human reproductive tract and 28-day menstrual cycle. Nat. Commun. 8, (2017).
5. Oleaga, C. et al. Investigation of the effect of hepatic metabolism on off-target cardiotoxicity in a multi-organ human-on-a-chip system. Biomaterials 182, 176-190 (2018).
6. Kimura, H., Yamamoto, T., Sakai, H., Sakai, Y. & Fujii, T. An integrated microfluidic system for long-term perfusion culture and on-line monitoring of intestinal tissue models. Lab. Chip 8, 741 (2008).
7. Jung, D. J. et al. A one-stop microfluidic-based lung cancer organoid culture platform for testing drug sensitivity. Lab. Chip 19, 2854-2865 (2019).
8. Wang, Y. et al. In situ differentiation and generation of functional liver organoids from human iPSCs in a 3D perfusable chip system. Lab. Chip 18, 3606-3616 (2018).
9. Shim, S., Belanger, M. C., Harris, A. R., Munson, J. M. & Pompano, R. R. Two-way communication between ex vivo tissues on a microfluidic chip: application to tumor-lymph node interaction. Lab. Chip 19, 1013-1026 (2019).
10. Ross, A. E. & Pompano, R. R. Diffusion of cytokines in live lymph node tissue using microfluidic integrated optical imaging. Anal. Chim. Acta 1000, 205-213 (2018).
11. Zhao, Y., Kankala, R., Wang, S.-B. & Chen, A.-Z. Multi-Organs-on-Chips: Towards Long-Term Biomedical Investigations. Molecules 24, 675 (2019).
12. Waheed, S. et al. 3D printed microfluidic devices: enablers and barriers. Lab. Chip 16, 1993-2013 (2016).
13. Maschmeyer, I. et al. A four-organ-chip for interconnected long-term co-culture of human intestine, liver, skin and kidney equivalents. Lab. Chip 15, 2688-2699 (2015).
14. Skardal, A. et al. Multi-tissue interactions in an integrated three-tissue organ-on-a-chip platform. Sci. Rep. 7, (2017).
15. Voet, V. S. D. et al. Biobased Acrylate Photocurable Resin Formulation for Stereolithography 3D Printing. ACS Omega 6 (2018).
16. Bhargava, K., Ermagan, R., Thompson, B., Friedman, A. & Malmstadt, N. Modular, Discrete Micromixer Elements Fabricated by 3D Printing. Micromachines 8, 137 (2017).
17. Au, A. K., Bhattacharjee, N., Horowitz, L. F., Chang, T. C. & Folch, A. 3D-printed microfluidic automation. Lab. Chip 15, 1934-1941 (2015).
18. Chu, C.-H. et al. Hybrid negative enrichment of circulating tumor cells from whole blood in a 3D-printed monolithic device. Lab. Chip 19, 3427-3437 (2019).
19. Bhatia, S. N. & Ingber, D. E. Microfluidic organs-on-chips. Nat. Biotechnol. 32, 760-772 (2014).
20. Munson, J. & Shieh, A. Interstitial fluid flow in cancer: implications for disease progression and treatment. Cancer Manag. Res. 317 (2014) doi:10.2147/CMAR.S65444.
21. Owens, C. E. & Hart, A. J. High-precision modular microfluidics by micromilling of interlocking injection-molded blocks. Lab. Chip 18, 890-901 (2018).
22. Ong, L. J. Y. et al. A pump-free microfluidic 3D perfusion platform for the efficient differentiation of human hepatocyte-like cells: Pump-Free Microfluidic 3D Perfusion Platform. Biotechnol. Bioeng. 114, 2360-2370 (2017).
23. Kusiak, J. L., Seevers, J. E. & Corporation, A. Centrifugal Water Pump. 8 (1990).
24. Tankut, A. N. & Tankut, N. The Effects of Joint Forms (Shape) and Dimensions on the Strengths of Mortise and Tenon Joints. Turkish Journal of Agriculture and Forestry 29, 493-498 (2005).

What is claimed is:

1. A system to model inter-organ communication comprising:
one or more micro-culture well configured to receive a tissue sample therein; and
an impeller-based pump in fluid communication with the one or more micro-culture well and configured to generate fluid flow through the one or more micro-culture well.

2. The system according to claim 1, wherein the one or more micro-culture well is configured to perfuse the fluid transversely with respect to the tissue sample.

3. The system according to claim 1, comprising a mesh base positioned within each of the one or more micro-culture well and configured to structurally support the tissue sample thereon.

4. The system according to claim 3, wherein the mesh base comprises one or more vertical posts that extend out of the one or more micro-culture well and are configured to aid in insertion and removal of the mesh base with respect to the one or more micro-culture well.

5. The system according to claim 3, comprising a membrane positioned between the tissue sample and the mesh base.

6. The system according to claim 1, wherein the impeller-based pump comprises:
an impeller positioned within a substantially circular chamber; and
a magnetic element coupled with the impeller;
wherein the impeller is rotatable within the substantially circular chamber upon application of a magnetic field to the magnetic element to generate fluid flow within the system.

7. The system according to claim 6, comprising a magnetic field generator spaced apart from the impeller-based pump, the magnetic field generator being configured to apply a rotating magnetic field to the magnetic element.

8. The system according to claim 1, wherein each of the one or more micro-culture well is contained within a sample container having a sample container inlet and a sample container outlet;
wherein the impeller-based pump comprises a pump inlet and a pump outlet; and
wherein each sample container and the pump module are arranged to form a single closed fluid circuit in which fluid is flowed into each respective one of the sample container inlet and the pump inlet and out of each respective one of the sample container outlet and the pump outlet.

9. The system according to claim 8, comprising one or more fluid conduit elements arranged to provide fluid connections among and between the impeller-based pump and the one or more sample container.

10. A method for modeling inter-organ communication, the method comprising:
positioning a live tissue sample or model thereof into each of one or more micro-culture well;
pumping fluid through the one or more micro-culture well.

11. The method according to claim 10, wherein pumping fluid through the one or more micro-culture well comprises perfusing the fluid transversely with respect to the live tissue sample or model thereof.

12. The method according to claim 10, wherein positioning a live tissue sample or model thereof into each of one or more micro-culture well comprises arranging a plurality of micro-culture wells in sequence within a single closed fluid circuit.

13. The method according to claim 10, wherein pumping fluid through the one or more micro-culture well comprises:
positioning an impeller-based pump in fluid communication with the one or more micro-culture well, the impeller-based pump comprising an impeller and a magnetic element coupled with the impeller; and
applying a rotating magnetic field to the magnetic element to rotate the impeller and generate fluid flow through the one or more micro-culture well.

14. The method according to claim 13, wherein applying a rotating magnetic field comprises activating a magnetic field generator that is spaced apart from the impeller-based pump.
